(19) United States
(12) Patent Application Publication
(10) Pub. No.: US 2026/0295282 A1
(43) Pub. Date: Oct. 1, 2026

(54) MAGNETIC INDUCTION ANTENNA MODULE

(71) Applicant: Cochlear Limited, Macquarie University, NSW (AU)

(72) Inventors: Guilhem Yvan VAVELIN, Nice (FR); Peter John RUSSELL, Regentville (AU); William WIDJAJA, North Ryde (AU); Roeland HUYS, Herent (BE); Padraig HURLEY, Frenches Forest (AU)

(21) Appl. No.: 19/477,327

(22) PCT Filed: Apr. 25, 2024
(86) PCT No.: PCT/IB2024/054059

§ 371 Date: Oct. 21, 2025

Related U.S. Application Data
(60) Provisional application No. 63/499,611, filed on May 2, 2023.

(51) Int. Cl. A61N 1/378; A61N 1/36; H01Q 7/00; H02J 50/10; H04R 25/00

(52) U.S. Cl. CPC A61N 1/3787; A61N 1/36038; H01Q 7/00; H02J 50/10; H04R 25/554; H04R 25/602; H04R 2225/021; H04R 2225/31; H04R 2225/51; H04R 2225/57; H04R 2225/67

(57) ABSTRACT

Presented herein is a removable battery pack that includes a housing, a battery disposed within the housing, and a magnetic induction (MI) antenna integrated in the housing and disposed adjacent to the battery, and is a modular behind-the-ear (BTE) device comprising: a sound processor configured to connect with a radio frequency (RF) coil; and a removable battery module configured to connect with the sound processor, wherein the removable battery module includes a battery and the MI antenna.

### FIELD OF THE INVENTION

The present invention relates generally to a magnetic induction (MI) antenna, and more specifically, to integrating the MI antenna in a battery module.

### RELATED ART

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a modular battery pack is provided. The modular battery pack comprises: a housing; a battery disposed within the housing; and a magnetic induction (MI) antenna integrated in the housing and disposed adjacent to the battery.

In another aspect, a modular behind-the-ear (BTE) device is provided. The modular BTE device comprises: a sound processor configured to connect with a radio frequency (RF) coil; and a removable battery module configured to connect with the sound processor, wherein the removable battery module includes a battery and a magnetic induction (MI) antenna.

In another aspect, a device is provided. The device comprises: at least one processor; a radio frequency (RF) coil configured to be electrically connected to the at least one processor; and a removable battery module configured to be electrically connected to the at least one processor, wherein the removable battery module includes at least one battery and a magnetic induction (MI) antenna disposed adjacent the battery.

In another aspect, a method is provided. The method comprises: at start-up of a sound processor of a modular behind-the-ear (BTE) device: determining an identifier of a battery module connected to the sound processor, wherein the battery module includes a battery and a magnetic induction (MI) antenna; and recalibrating one or more parameters associated with the MI antenna based on the identifier of the battery module.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device configured to operate with the cochlear implant system of FIG. 1A;

FIGS. 2A and 2B show an example system that includes a sound processor and a battery module connected with the sound processor;

FIG. 3A is a diagram of a system that includes a sound processor and a battery module (collectively referred to as a “modular BTE” device), according to an example embodiment;

FIGS. 3B and 3C are circuit diagrams relating to a first concept for the modular BTE device of FIG. 3A, which involves substituting existing I2C pins of the interface connector for MI antenna connections and multiplexing the MI antenna signal, according to an example embodiment;

FIGS. 3D and 3E are circuit diagrams relating to a second concept for the modular BTE device of FIG. 3A, which involves adding two new dedicated pins to the interface connector for the MI antenna connections, according to another example embodiment;

FIGS. 4A and 4B are diagrams of a system that includes a sound processor, an RF transmitter, and a battery module, according to an example embodiment;

FIG. 4C is a circuit diagram for the sound processor and the battery module of FIGS. 4A and 4B;

FIG. 5 is a flowchart illustrating a method for recalibrating MI antenna parameter(s) at each boot/start-up of a sound processor of a modular BTE device, according to an example embodiment; and

FIG. 6 is a schematic diagram illustrating a vestibular stimulator system with which aspects of the techniques presented herein can be implemented.

### DETAILED DESCRIPTION

### Overview

Presented herein are techniques for locating a magnetic induction (MI) antenna of an electronic device, used for MI communication/MI link(s), in a removable battery module (e.g., rechargeable and/or replaceable battery pack) of the electronic device. In certain embodiments, the electronic device is a behind-the-ear (BTE) sound processor comprising at least one inductive link radio-frequency (RF) coil for closely-coupled inductive communication (RF communication/RF link). In such embodiments, integration of the MI antenna in the removable battery module sufficiently separates the MI antenna from the RF coil(s) of the sound processor (and the coils of the associated cochlear implant) to protect the MI communication from interference associated with the closely-coupled inductive communication.

For a BTE sound processor, integrating an MI antenna into the removable battery module can maximize the distance between the RF coil and the MI antenna to reduce interference. In certain examples, the MI antenna is then used to support ear-to-ear communication for bilateral users, which provides advantages such as synchronizing volume and program settings, or sharing microphone data for more advanced signal processing, including but not limited to bilateral beamforming. Providing a battery module for the sound processor with an MI antenna integrated therein can improve functioning and performance of the magnetic induction link (due to the reduced interference between the RF coil and the MI antenna), and also enhances ear-to-ear communication using the magnetic induction link of the MI antenna. Additionally, integrating the MI antenna in the battery module can provide improved shielding via the casing (e.g., an aluminum casing) of the battery module.

As noted, there are a number of different types of electronic devices in/with which the techniques presented herein can be implemented. Merely for ease of description, the techniques presented herein are primarily described with reference to a specific electronic device in the form of a cochlear implant system. However, it is to be appreciated that the techniques presented herein can also be partially or fully implemented by any of a number of different types of electronic devices, including consumer electronic devices (e.g., mobile devices, wearables, computing devices, televisions, appliances/white goods, etc.), other medical devices, diagnostic equipment, etc. For example, the techniques presented herein could be implemented by hearing devices, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. In further embodiments, the techniques presented herein can be implemented with air purifiers or air sensors (e.g., automatically adjust depending on environment), hospital beds, identification (ID) badges/bands, or other hospital equipment or instruments, or the like.

As used herein, the term “hearing device” is to be broadly construed as any device that delivers sound signals to a user in any form, including in the form of acoustical stimulation, mechanical stimulation, electrical stimulation, optical stimulation, etc. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems. combinations or variations thereof, etc.) or a device for use by a person with normal hearing (e.g., consumer devices that provide audio streaming, consumer headphones, earphones, and other listening devices).

### Example System and Devices

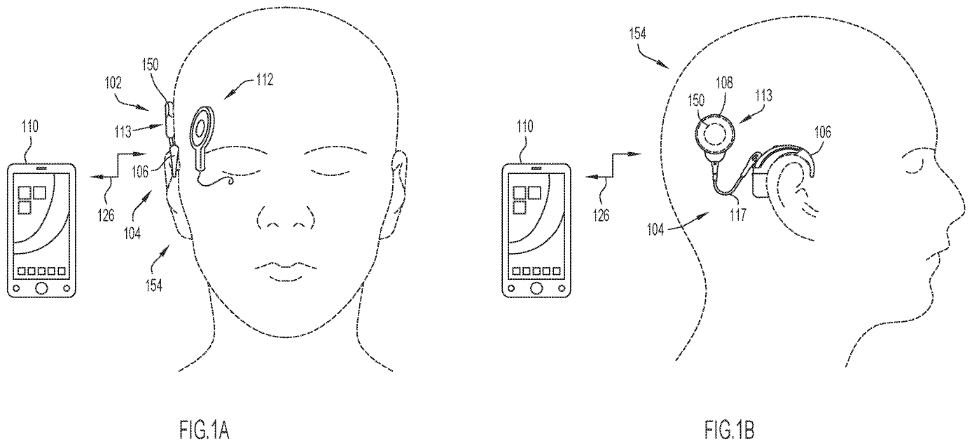

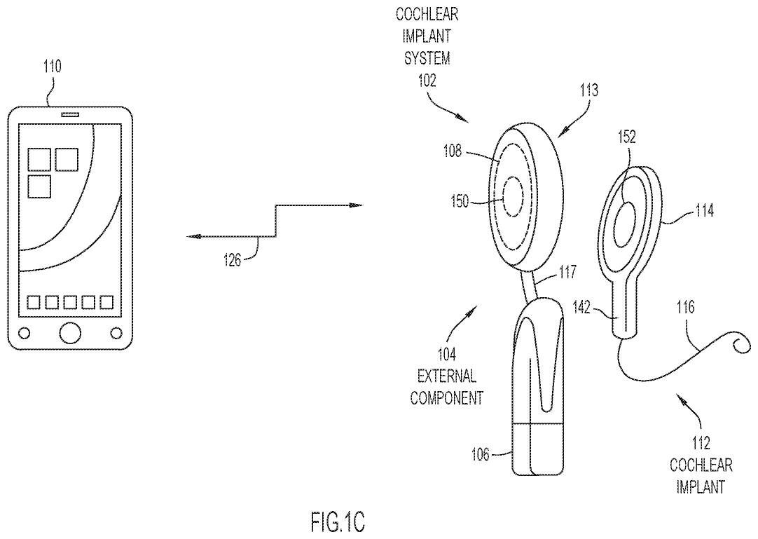

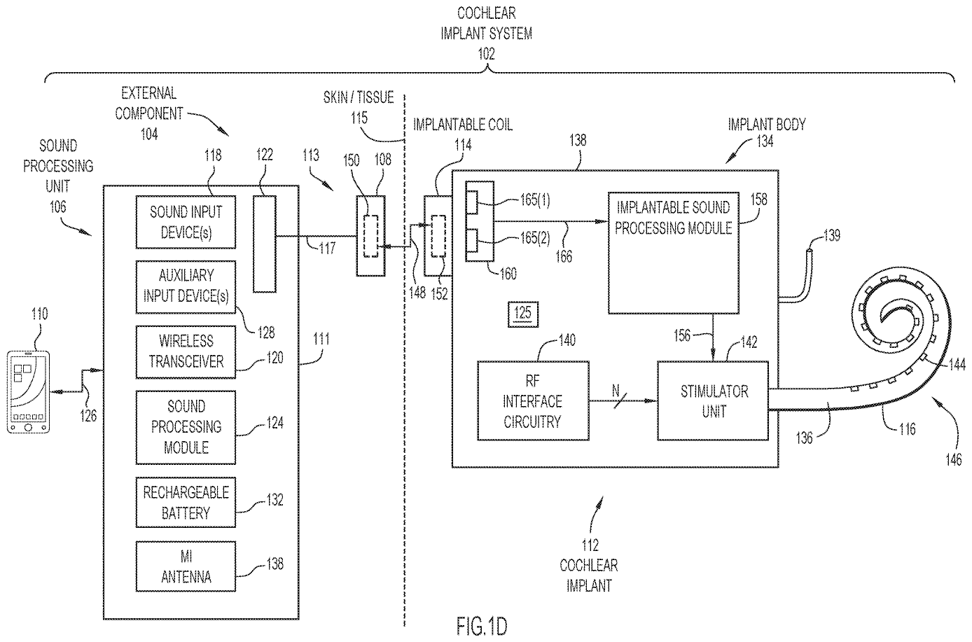

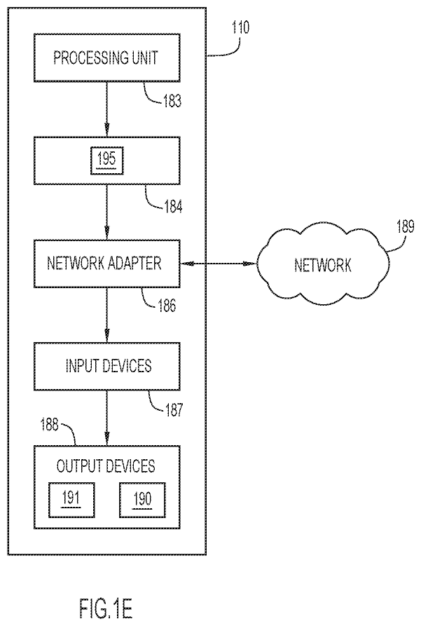

FIGS. 1A-1D illustrates an example cochlear implant system 102 with which aspects of the techniques presented herein can be implemented. The cochlear implant system 102 comprises an external component 104 that is configured to be directly or indirectly attached to the body of the user, and an internal/implantable component 112 that is configured to be implanted in or worn on the head of the user. In the examples of FIGS. 1A-1E, the implantable component 112 is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

In the examples of FIGS. 1A-ID, the external component 104 comprises a sound processing unit 106 and a separate external RF coil assembly 113. The cochlear implant 112 includes an implantable coil 114, an implant body 134, and an elongate stimulating assembly 116 configured to be implanted in the user's cochlea.

In one example, the sound processing unit 106 is a behind-the-ear (BTE) sound processing unit, sometimes referred to herein as an BTE sound processor or BTE component, that is configured to send data and power to the implantable component 112. In general, a BTE sound processing unit 106 is a component having a housing 111 and that is shaped to be worn on the outer ear of the user and is connected to the separate external RF coil assembly 113 via a cable 117. The external RF coil assembly 113 is configured to be magnetically and inductively coupled to the implantable coil 114. In particular, the external RF coil assembly 113 comprises a magnet 150 configured to be magnetically coupled to an internal/implantable magnet 152 in the implantable component 112). The external RF coil assembly 113 also includes an external (headpiece) coil 108 (the external RF coil 108) that is configured to be inductively coupled to the implantable coil 114.

It is to be appreciated that the BTE sound processing unit 106 is merely illustrative of the external devices that could operate with implantable component 112. For example, in alternative examples, the external component 104 may comprise an off-the-ear (OTE) sound processing unit.

Although the cochlear implant system 102 includes the sound processing unit 106 and the cochlear implant 112, as described below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110, which is shown in greater detail in FIG. 1E, is a computing device, such as a personal computer (e.g., laptop, desktop, tablet), a mobile phone (e.g., smartphone), remote control unit, etc. The external device 110 and the cochlear implant system 102 (e.g., sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the sound processing unit 106 of the external component 104 also comprises one or more input devices configured to capture and/or receive input signals (e.g., sound or data signals) at the sound processing unit 106. The one or more input devices include, for example, one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a short-range wireless transmitter/receiver (wireless transceiver) 120 (e.g., for communication with the external device 110), each located in, on or near the sound processing unit 106. However, it is to be appreciated that one or more input devices may include additional types of input devices and/or less input devices (e.g., the short-range wireless transceiver 120 and/or one or more auxiliary input devices 128 could be omitted).

The sound processing unit 106 also comprises a closely-coupled radio frequency transmitter/receiver (RF transceiver) 122, an external sound processing module 124, and a removable battery module 130. The removable battery module 130 includes at least one rechargeable battery 132 and a magnetic induction (MI) antenna 138. As described further below, the removable battery module 130 is a removable component.

The external sound processing module 124 can be configured to perform a number of operations, and can be formed by one or more processors (e.g., one or more Digital Signal Processors (DSPs), one or more uC cores, etc.), firmware, software, etc. arranged to perform operations described herein. That is, the external sound processing module 124 can be implemented as firmware elements, partially or fully implemented with digital logic gates in one or more application-specific integrated circuits (ASICs), partially or fully in software, etc.

Returning to the example of FIGS. 1A-1D, the implantable component 112 comprises an implant body (main module) 134, a lead region 136, and the intra-cochlear stimulating assembly 116, all configured to be implanted under the skin (tissue) 115 of the user. The implant body 134 generally comprises a hermetically-sealed housing 138 that includes, in certain examples, at least one power source 125 (e.g., one or more batteries, one or more capacitors, etc.) 125, in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact array (electrode array) 146 for delivery of electrical stimulation (current) to the recipient's cochlea. Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external RF coil 108 and the implantable coil 114. The external magnet 150 is fixed relative to the external RF coil 108 and the internal/implantable magnet 152 is fixed relative to the implantable coil 114. The external magnet 150 and the internal/implantable magnet 152 fixed relative to the external RF coil 108 and the internal/implantable coil 114, respectively, facilitate the operational alignment of the external RF coil 108 with the implantable coil 114. This operational alignment of the coils enables the external component 104 to transmit data and power to the implantable component 112 via a closely-coupled wireless link 148 formed between the external RF coil 108 with the implantable coil 114. In certain examples, the closely-coupled wireless link 148 is a radio frequency (RF) link. However, various other types of energy transfer, such as infrared (IR), electromagnetic, capacitive, and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to process the received input audio signals (received at one or more of the input devices, such as sound input devices 118 and/or auxiliary input devices 128), and convert the received input audio signals into output control signals for use in stimulating a first ear of a recipient or user (i.e., the external sound processing module 124 is configured to perform sound processing on input signals received at the sound processing unit 106). Stated differently, the one or more processors (e.g., processing element(s) implementing firmware, software, etc.) in the external sound processing module 124 are configured to execute sound processing logic in memory to convert the received input audio signals into output control signals (stimulation signals) that represent electrical stimulation for delivery to the recipient.

As noted, FIG. 1D illustrates an embodiment in which the external sound processing module 124 in the sound processing unit 106 generates the output control signals. In an alternative embodiment, the sound processing unit 106 can send less processed information (e.g., audio data) to the implantable component 112 and the sound processing operations (e.g., conversion of input sounds to output control signals 156) can be performed by a processor within the implantable component 112.

In FIG. 1D, according to an example embodiment, output control signals (stimulation signals) are provided to the RF transceiver 122, which transcutaneously transfers the output control signals (e.g., in an encoded manner) to the implantable component 112 via external RF coil 108 and implantable coil 114. That is, the output control signals (stimulation signals) are received at the RF interface circuitry 140 via implantable coil 114 and provided to the stimulator unit 142. The stimulator unit 142 is configured to utilize the output control signals to generate electrical stimulation signals (e.g., current signals) for delivery to the user's cochlea via one or more of the stimulating contacts (electrodes) 144. In this way, cochlear implant system 102 electrically stimulates the user's auditory nerve cells, bypassing absent or defective hair cells that normally transduce acoustic vibrations into neural activity, in a manner that causes the recipient to perceive one or more components of the input audio signals (the received sound signals).

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, an example embodiment of the cochlear implant 112 can include a plurality of implantable sound sensors 165(1), 165(2) that collectively form a sensor array 160, and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 165(1), 165(2) of the sensor array 160 are configured to detect/capture input sound signals 166 (e.g., acoustic sound signals, vibrations, etc.), which are provided to the implantable sound processing module 158. The implantable sound processing module 158 is configured to convert received input sound signals 166 (received at one or more of the implantable sound sensors 165(1), 165(2)) into output control signals 156 for use in stimulating the first ear of a recipient or user (i.e., the implantable sound processing module 158 is configured to perform sound processing operations). Stated differently, the one or more processors (e.g., processing element(s) implementing firmware, software, etc.) in implantable sound processing module 158 are configured to execute sound processing logic in memory to convert the received input sound signals 166 into output control signals 156 that are provided to the stimulator unit 142. The stimulator unit 142 is configured to utilize the output control signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 165(1), 165(2) of sensor array 160 in generating stimulation signals for delivery to the user.

FIG. 1E is a block diagram illustrating one example arrangement for an external computing device 110 configured to perform one or more operations in accordance with certain embodiments presented herein. As shown in FIG. 1E, in its most basic configuration, the external computing device 110 includes at least one processing unit 183 and a memory 184. The processing unit 183 includes one or more hardware or software processors (e.g., Central Processing Units) that can obtain and execute instructions. The processing unit 183 can communicate with and control the performance of other components of the external computing device 110. The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include random access memory (RAM), read only memory (ROM), EEPROM (Electronically-Erasable Programmable Read-Only Memory), flash memory, optical disc storage, magnetic storage, solid state storage, or any other memory media usable to store information for later access. By way of example, and not limitation, the memory 184 can include wired media such as a wired network or direct-wired connection, and wireless media such as acoustic, RF, infrared and other wireless media, or combinations thereof. In certain embodiments, the memory 184 comprises logic 195 that, when executed, enables the processing unit 183 to perform aspects of the techniques presented.

In the illustrated example of FIG. 1E, the external computing device 110 further includes a network adapter 186, one or more input devices 187, and one or more output devices 188. The external computing device 110 can include other components, such as a system bus, component interfaces, a graphics system, a power source (e.g., a battery), among other components. The network adapter 186 is a component of the external computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. The one or more input devices 187 are devices over which the external computing device 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the computing device 110 is able to provide output to a user. The output devices 188 can include, for example, a display 190 (e.g., a liquid crystal display (LCD)) and one or more speakers 191, among other output devices for presentation of visual or audible information to the recipient, a clinician, an audiologist, or other user.

It is to be appreciated that the arrangement for the external computing device 110 shown in FIG. 1E is merely illustrative and that aspects of the techniques presented herein can be implemented at a number of different types of systems/devices including any combination of hardware, software, and/or firmware configured to perform the functions described herein. For example, the external computing device 110 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

Certain implantable medical devices use a closely-coupled inductive link to convey power, and potentially data, from an external device to an implantable component. One technology that is often used to transmit power (and potentially data) is the near electromagnetic field. However, this type of link (also referred to as a radio frequency (RF) link herein) creates harmonics and spurious emissions that can easily be picked up by a weak magnetic receiver, such as a telecoil or a magnetic induction (MI) antenna operating as part of an MI link. That is, RF links produce relatively strong magnetic fields that can interfere with an MI link.

There is an increased use of bilateral communications (e.g., bilateral beamforming) where two hearing devices, each worn at a different ear, are communicating with each other (e.g., to generate this bilateral beamforming to improve patient audio experience, especially within a noisy environment. Since an MI link can be used to for bilateral communication (also known as ear-to-ear or e2e communication), this MI link will be sensitive to the existing RF link. For BTE sound processors, deciding where to locate the MI antenna is complicated when it comes to designing a “modular BTE” sound processor for which the rechargeable battery pack is removable and replaceable. For example, there is not much space left in the casing to fit the MI antenna, and there may not be enough clearance between the MI antenna and the RF coil, which may result in interference.

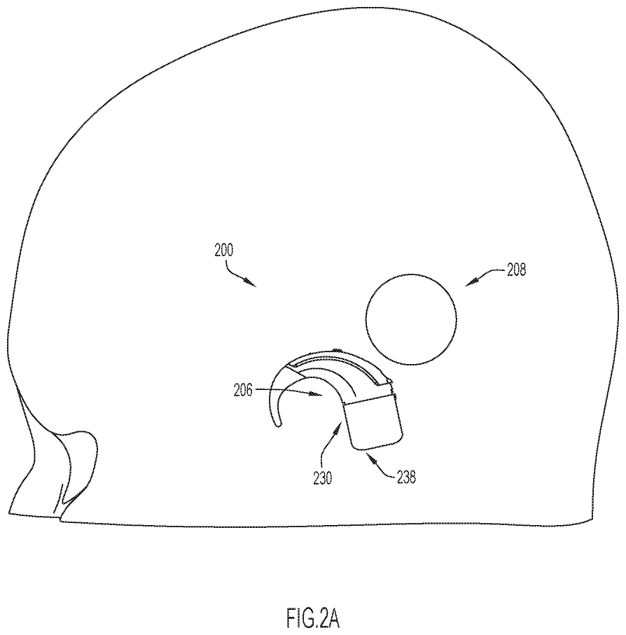

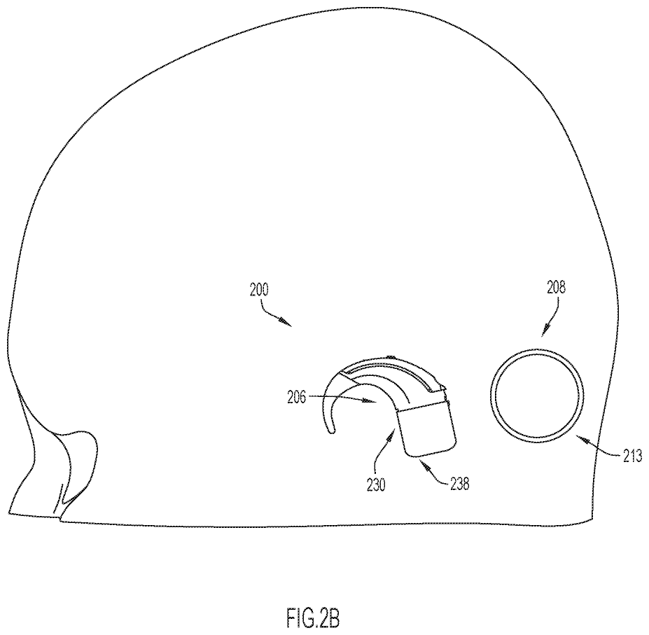

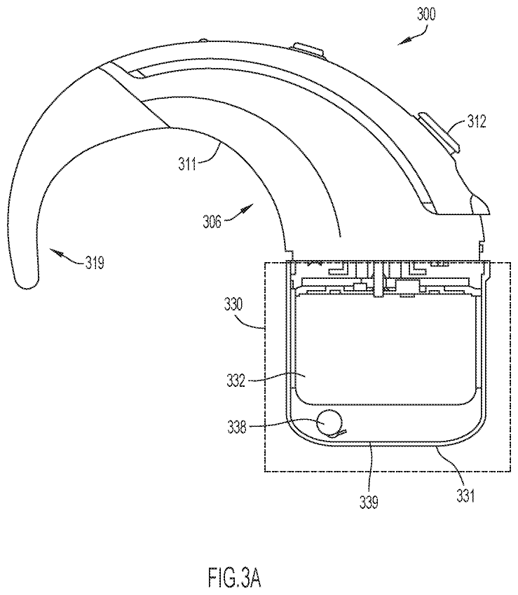

A sufficient separation distance (also referred to herein as “clearance”) is needed in order to improve the coexistence between the RF link and the MI link (e.g., sufficient distance between the stronger RF coil and the weaker MI antenna). FIGS. 2A and 2B show an example system 200 that includes a BTE sound processor 206 and a battery module 230 connected with the BTE sound processor 206. The BTE sound processor 206 and the battery module 230 may also be referred to collectively as a “modular BTE” device herein. The battery module 230 includes an MI antenna 238, which may be disposed below the battery of the battery module 230, for example. The system 200 further includes an external RF coil assembly 213 that connects with the sound processor 206 and includes an external RF coil 208. In FIGS. 2A and 2B, the thin circle represent the external RF coil 208 that is connected with the sound processor 206.

It has been determined that a distance of approximately 3 cm (or 30 mm) between the MI antenna 238 of the battery module 230 and the external RF coil 208 of the external RF coil assembly 213 is the minimum separation distance that is needed to ensure a good coexistence between the RF link and the MI link. As shown in FIG. 2A, when the modular BTE device (i.e., the sound processor 206 and the battery module 230) is positioned over the pina of the recipient, the modular BTE device allows the MI antenna 238 (in the battery module 230) to be properly positioned and to keep a clearance of at least 3 cm (or 30 mm) between the external RF coil 208 and thus the associated implantable coil.

In some instances, the clearance between the external RF coil 208 and the MI antenna 238 may be increased to approximately 4 cm (or 40 mm). FIG. 2B shows an example external RF coil 208, and an additional 10 mm radius to account for a clearance of approximately 4 cm (or 40 mm) clearance between the MI antenna 238 and the external RF coil 208.

New surgical guidance for MI capable implant location is to use a minimum distance of 4 cm (40 mm) between the ear canal and the implant lead exit, which should remove any dead zone. The minimum clearance of 4 cm (40 mm) corresponds to the closest position of the implant to the ear canal, according to the surgical guidance. There may also be a maximum clearance of 6 cm (60 mm), which corresponds to the maximum distance of electrode strain relief from the ear canal (based on electrode lead length).

MI antenna placement in the case of a bilateral MI link for certain implants should preferably meet the specified minimum clearance between the RF coil and the MI antenna (e.g., approximately 3 cm for a split RF link, or approximately 4 cm for a combined RF link). For a modular BTE with a specification of at least 4 cm clearance between the external RF coil 208 and the MI antenna 238, for example, locating the MI antenna 238 in the bottom of the battery module 230 may provide the best coverage for a majority of recipients. If the MI antenna 238 were integrated in the sound processor 206 (instead of the battery module 230), the MI clearance may not be acceptable for many recipients.

Thus, in the case of certain implants (e.g., a modular BTE sound processor) with a combined RF link and a bilateral MI link, the minimum clearance required between the MI antenna and the RF coil is approximately 4 cm (or 40 mm). In other cases, such as, smart implants or BTE devices with a split RF link and a bilateral MI link, or monolithic BTE devices or other future implants with MI capability that have a separated RF link and both ipsilateral and bilateral MI links, the minimum clearance required between the MI antenna and the RF coil is approximately 3 cm (or 30 mm).

Accordingly, an example embodiment places/integrates the MI antenna 238 into the modular battery pack 230 of the BTE sound processor. This location may be preferable to placement in other parts of the sound processor 206, in order to minimize interference.

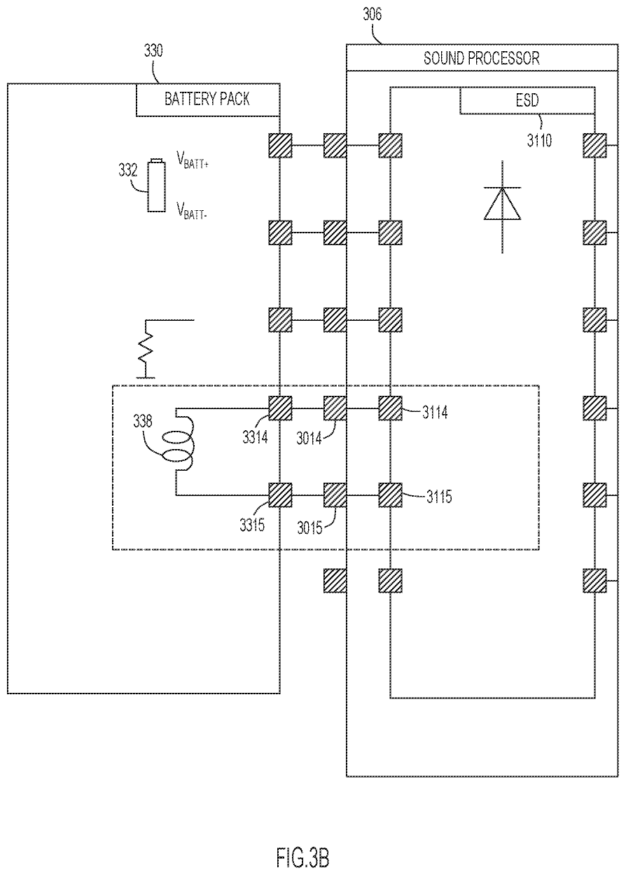

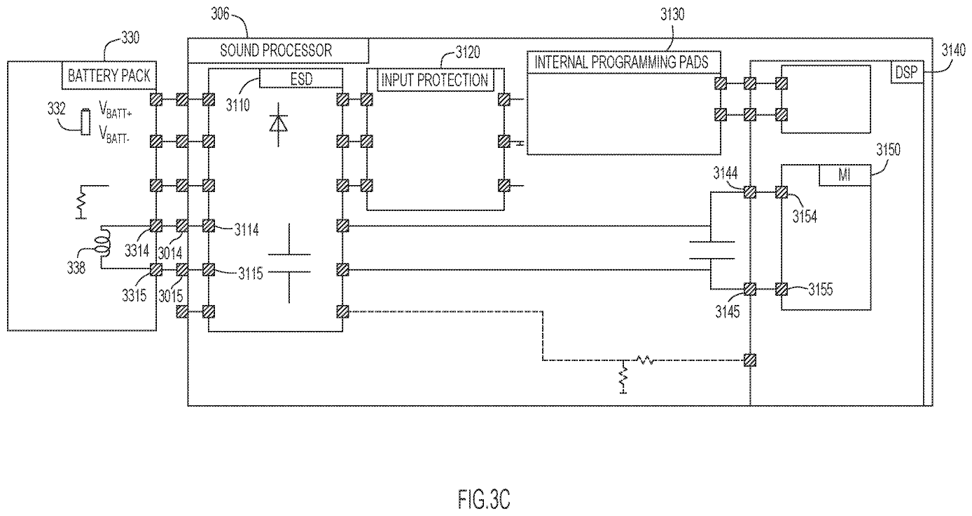

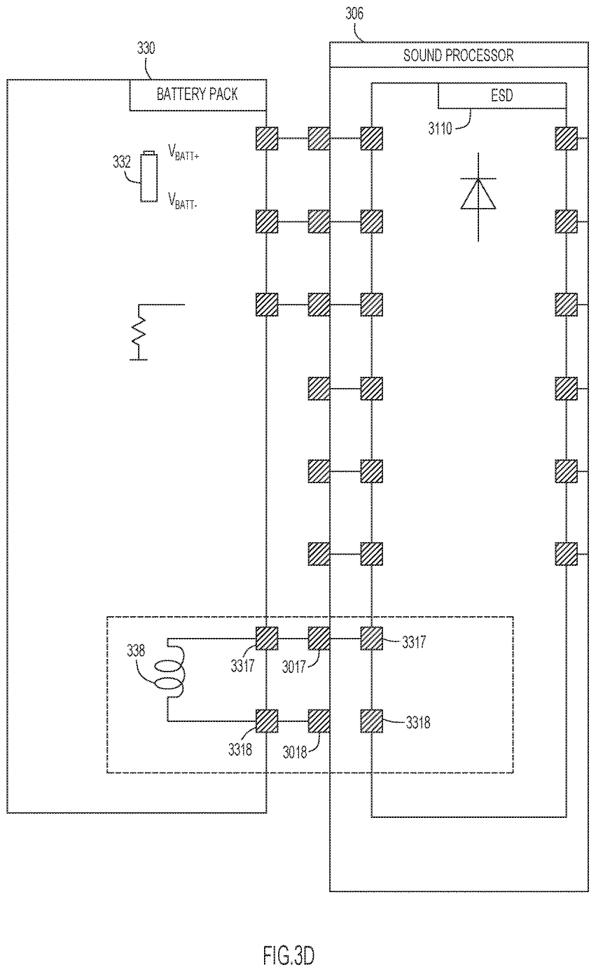

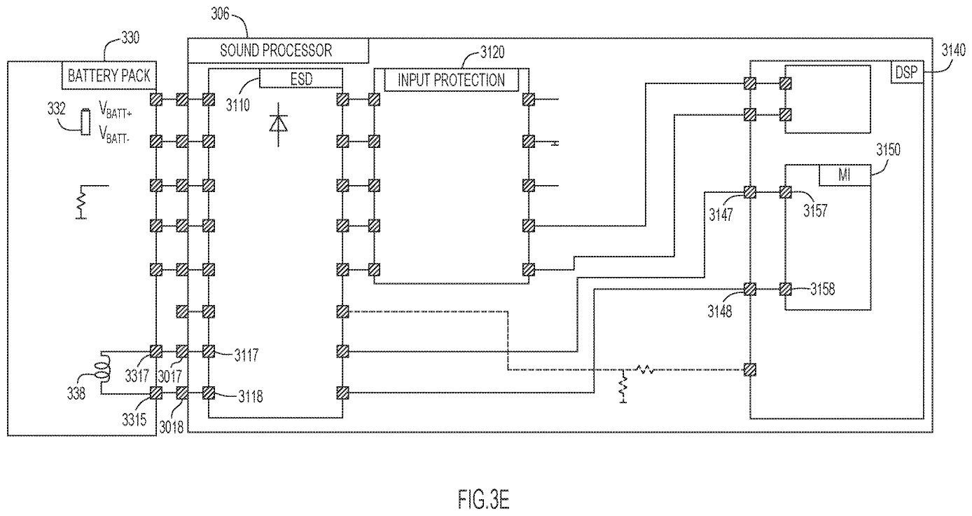

As mentioned above, a preferred example embodiment provides a modular BTE sound processor that has an MI antenna embedded under the rechargeable battery, such that the MI antenna is made part of the modular battery pack, as depicted in FIG. 3A.

FIG. 3A is a diagram of a system 300 that includes a BTE sound processor 306 and a battery module 330 (collectively referred to as a “modular BTE” device), according to an example embodiment. The sound processor 306 may include a housing 311, an RF port 312, and an earhook 319, among various other components as described further below. Although not shown in FIG. 3A, the system 300 may further include an RF transceiver coupled to an external RF coil, which connects to the RF port 312 of the sound processor 306 via an RF connector.

The battery module 330 may include a housing 331, a battery 332, and an MI antenna 338. The housing 331 is an enclosure such as an aluminum casing, for example. The battery 332 may be removable, rechargeable, and replaceable. The sound processor 306 and the battery module 330 also include various circuitry (e.g., voltage, battery ID, protection components, communication components, etc.), which will be described below with reference to FIGS. 3B and 3C (first concept) and FIGS. 3D and 3E (second concept).

In some example embodiments, the housing 331 may include an extension 339 that extends below the battery 332, where the extension 339 provides extra space within the housing 331 to accommodate the MI antenna 338 below the battery 332. Preferably, the extension 339 is disposed at one side (e.g., lower left corner in FIG. 3A) of the battery module 330 and the housing 331 that is located opposite from the RF port 312 of the sound processor 306, such that the extension 339 and hence the MI antenna 338 are located as far away from the RF coil 328 of the RF transmitter 320 as possible. It is noted that the specific size and shape of the extension 339 is not limited to the illustrative example shown in FIG. 3A, and various other configurations for the extension 339 are also possible.

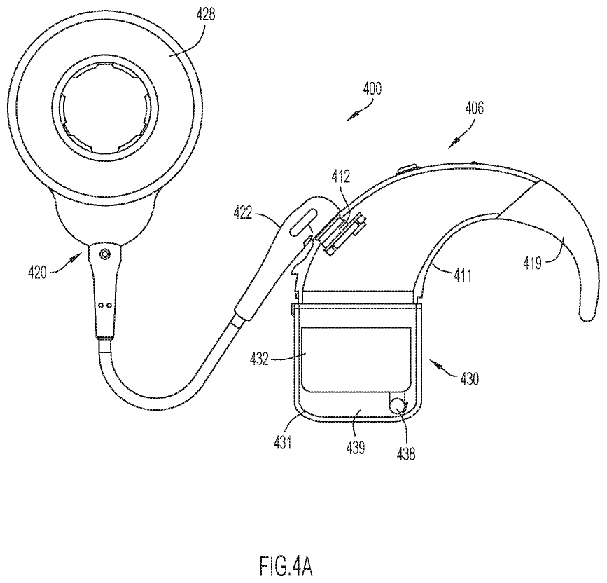

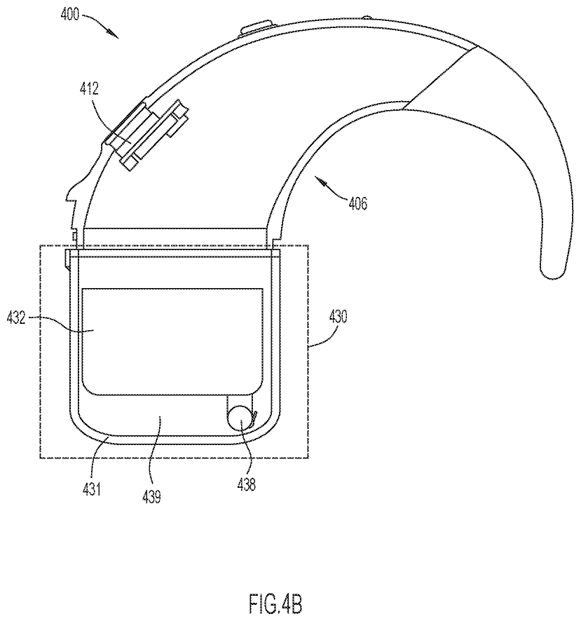

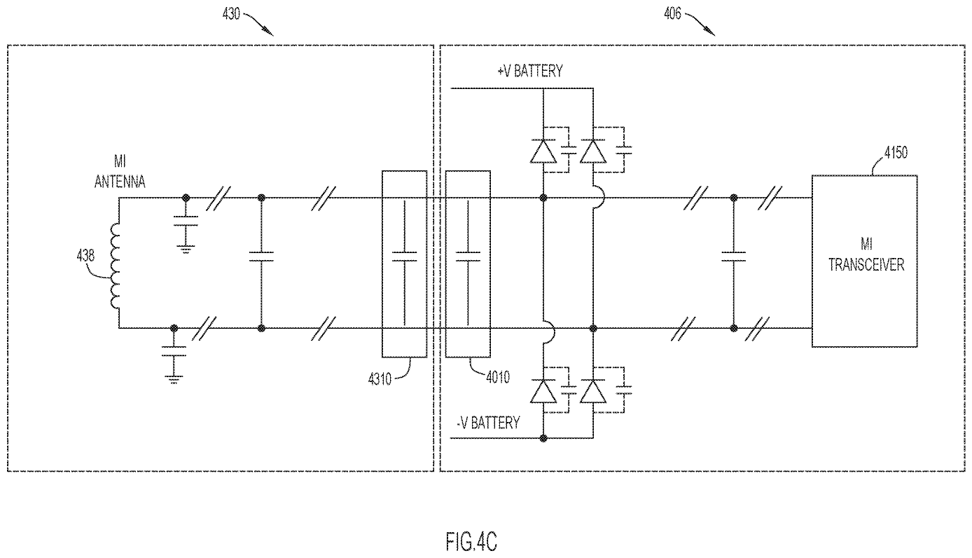

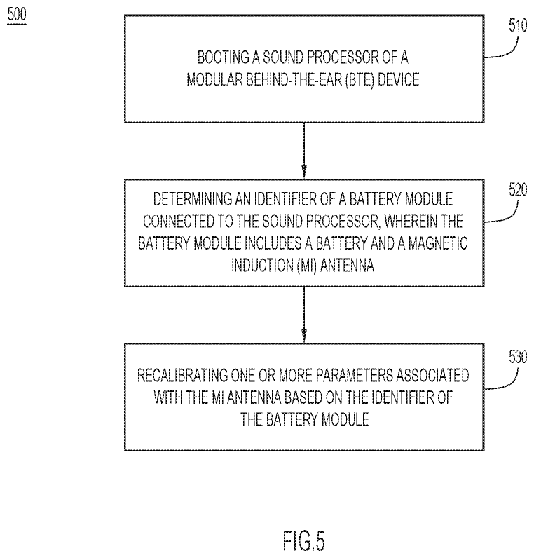

Preferably, the MI antenna 338 is disposed at least 3 cm apart from the RF coil 328 (not shown in FIG. 3A, refer to RF coil 428 of FIG. 4A) that will connect to the RF port 312 of the sound processor 306. As stated above, this has been determined to be the minimum separation distance that is needed for reducing interference and ensuring good coexistence between the RF link and the MI link. In some instances, the minimum clearance required between the MI antenna 338 and the RF coil 328 may be 4 cm (e.g., for legacy implants with a combined RF link and a bilateral MI link).

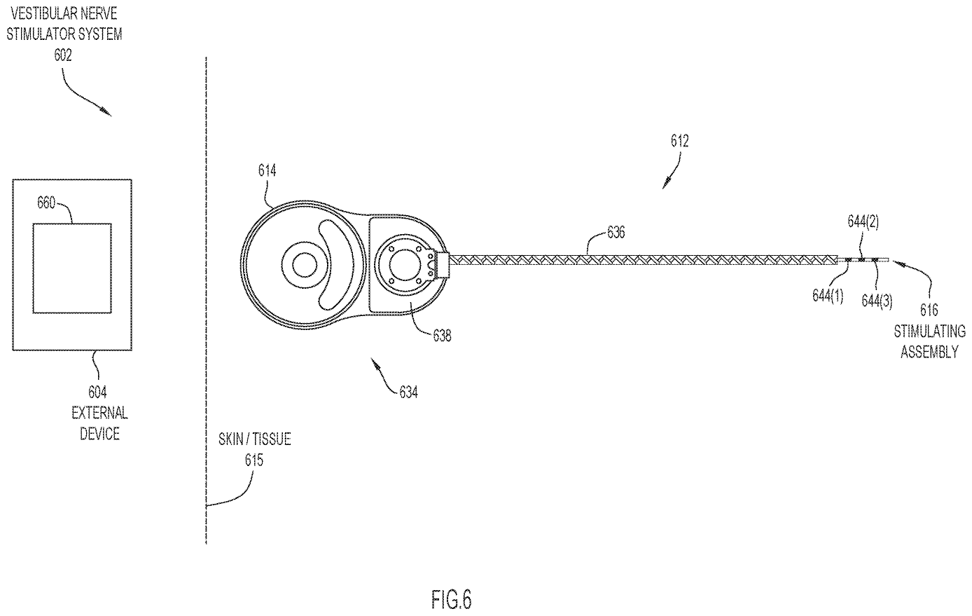

Various other technical issues are also resolved by the circuitry designs described below with reference to FIGS. 3B-3E. There are several challenges/risks to consider, and corresponding responses/mitigations are provided below:
(1) There is a need to protect the MI antenna lines from electrostatic discharge (ESD) introduced by exposing the MI antenna port to the outside world (the environment) because of the interface connector. One solution is to add one ESD protection diode to each antenna line, at the risk of creating parasitic capacitance seen by the MI chip.
(2) There is also a need to adapt to variable parasitic conditions. This issue is linked to the fact that the recipient could swap their battery according to their charge level, which may lead to having potentially different tuning parameters linked to different parasitic conditions. One solution is to re-run an antenna matching at each start-up to make sure the antenna is always matched whenever the battery module is swapped with another one (e.g., refer to method 500 of FIG. 5).
(3) There is a further need to assess the potential effect of the battery itself on the MI link quality. Providing some shielding for the MI antenna could impact antenna parameters (e.g., inductance value and Q value), but the shielding may be beneficial to attenuate some unwanted electromagnetic field noise, especially coming from the sound processor itself.

### Interface Connector Options for Combined MI Antenna & Battery Module

Next, different concepts for providing a modular BTE sound processor with an MI antenna integrated in the battery pack are described with reference to FIG. 3B-3E.

FIGS. 3B and 3C are circuit diagrams relating to a first concept for the modular BTE device of FIG. 3A, according to an example embodiment. The first concept of FIGS. 3B and 3C is to substitute existing I2C pins of the interface connector for MI antenna connections, and involves multiplexing the MI antenna signal (provide MI link) on the I2C communication lines.

As shown in FIGS. 3B and 3C, the battery pack 330 includes the battery 332 and the MI antenna 338, and the sound processor 306 includes an electrostatic discharge module 3110 (ESD). Each of the sound processor 306 and the battery pack 330 has an interface connector including a number of pins. In particular, sound processor 306 includes I2C pins 3014, 3015, and ESD 3110 includes I2C pins 3113, 3115. In battery pack 330, the MI antenna 338 is connected to pins 3314, 3315 (MI+ and MI−). As shown in FIG. 3C, the sound processor 306 further includes an input protection module 3120, internal programming pads 3130, and a digital signal processing module 3140 (DSP), where the DSP module 3140 includes a magnetic induction transceiver 3150 (MI). The I2C pins 3114, 3115 of ESD 3110 are connected with pins 3144, 3145 of DSP module 3140, which are further connected with pins 3154, 3155 (ANT1 and ANT2) of MI transceiver 3150. Thus, the MI antenna signal can be transmitted from the MI antenna 338 of battery pack 330 to the MI transceiver 3150 of sound processor 306 via the above-described I2C pins and corresponding connections which provide I2C communication lines.

The first concept of FIGS. 3B and 3C retains the existing 6 pin interface connector in the sound processor (no mechanical redesign needed). Potential drawbacks include a high MI signal loss due to the multiplexing operations, a firmware/software update to support MI while in programming mode, and two additional multiplexers in the sound processor.

The first concept may benefit from an assessment regarding whether MI antenna can or cannot be tuned in the presence of parasitic capacitances and ESD diodes capacitances, and/or the extent to which such MI antenna tuning is needed for the specific case of a sound processor of a “modular BTE” device (as distinguished from a larger “monolithic BTE” device).

There may be a concern that changing the battery pack (e.g., to replace it with a charged battery pack) would also change the MI antenna tuning. The solution to this is to implement a process to recalibrate every time at boot of the BTE sound processor (e.g., refer to method 500 of FIG. 5), to ensure that the tuning of the MI transceiver 3150 in the sound processor 306 will always match the tuning of the MI antenna 338 in the battery module 330 connected thereto, even when the recipient swaps out multiple battery packs that may have slightly different MI antenna tuning.

There could also be MI antenna performance differences (e.g., a risk of creating a new dead zone) with different batches of batteries or different battery manufacturers. Possible mitigations include performing a process to identify different MI batteries (and make corresponding adjustments based on the battery ID, batch number, manufacturer, etc.), and/or increasing the length of the MI rod, for example.

Generally, the first concept of FIGS. 3B and 3C has a minimal impact from parasitics to MI antenna inputs. The SYNC pin is not required for accessory switching (but can be retained for sync output). This example only adds the MI antenna component itself in the battery module (and the multiplexers in the sound processor), which is a simpler design (e.g., compared to the second concept of FIGS. 3D and 3E, which adds new components via a structural redesign of the circuitry of the sound processor 306 and the battery module 330).

FIGS. 3D and 3E are circuit diagrams relating to a second concept for the modular BTE device of FIG. 3A, according to another example embodiment. The second concept of FIGS. 3D and 3E is to add two new pins (dedicated MI pins) to the interface connector for the MI antenna connections, and communicating the MI antenna signal (provide MI link) on dedicated MI communication lines.

As shown in FIGS. 3D and 3E, the battery pack 330 includes the battery 332 and the MI antenna 338, and the sound processor 306 includes an ESD 3110. Each of the sound processor 306 and the battery pack 330 has an interface connector including a number of pins. In particular, sound processor 306 includes dedicated MI pins 3017, 3018, and ESD 3110 includes dedicated MI pins 3117, 3118 (MI+ and MI−). In battery pack 330, the MI antenna 338 is connected to pins 3317, 3318 (MI+ and MI−). As shown in FIG. 3E, the sound processor 306 further includes an input protection module 3120 and a DSP module 3140, where the DSP module 3140 includes an MI transceiver 3150. The dedicated MI pins 3117, 3118 of ESD 3110 are connected with pins 3147, 3148 of DSP module 3140, which are further connected with pins 3157, 3158 (ANT1 and ANT2) of MI transceiver 3150. Thus, the MI antenna signal can be transmitted from the MI antenna 338 of battery pack 330 to the MI transceiver 3150 of sound processor 306 via the above-described dedicated MI pins and corresponding connections which provide dedicated MI communication lines.

The second concept of FIGS. 3D and 3E meets current MI signal requirements. There is no need to provide additional multiplexers in the sound processor for multiplexing the MI antenna signal on the I2C communication lines, since the battery module 330 and the sound processor 306 each have dedicated connections for the MI link. Potential drawbacks are that a larger 8 pin interface connector is required in the sound processor 306 (potentially increasing the size of both the sound processor and the battery module), and that adding two new dedicated MI pins necessitates a mechanical redesign of the existing interface connector. Nevertheless, the second concept (involving adding new dedicated pins for the MI link) may be preferable because it would result in lower MI signal loss compared to the first concept (which involves multiplexing operations).

Similar challenges/risks and corresponding responses/mitigations may apply for the second concept of FIGS. 3D and 3E as described above with respect to the first concept (e.g., regarding MI antenna tuning in the presence of parasitic capacitances and ESD diodes capacitances, a process for recalibrating every time at boot of the BTE sound processor to make adjustments in case exchanging battery packs changes the MI antenna tuning, a battery identification process to account for possible MI antenna performance differences, etc.).

A possible third concept (not shown in figures) involves sharing the battery + and − pins (V_{BATT} and VSSNEO) for the MI antenna signals (MI link), by capacitively coupling the MI antenna across the battery terminals. A possible fourth concept (not shown in figures) involves sharing the battery ID pins (BATTID) for the MI antenna signals (MI link). Sharing the battery pins for the MI antenna signals shows very low attenuation in electrical simulations on theoretical components. Likewise, sharing the BATTID pins maintains low MI signal attenuation. Additional steps may include further circuit simulation with off-the-shelf component models from datasheets and with dynamic sound processor loads.

The third and fourth concepts meet current MI signal requirements and retain the existing 3 pin interface connector in the battery module and the existing 6 pin interface connector in the sound processor. However, these alternative examples each require a number of additional components in both the sound processor and the battery module (e.g., a total of four components in the battery module and four components in the sound processor in the case of the third concept, or a total of two components in the battery module and two components in the sound processor in the case of the fourth concept). There may also be coexistence risks with respect to the power supply. Therefore, these possible additional concepts may be less preferred compared to both of the first concept of FIGS. 3B and 3C and the second concept of FIGS. 3D and 3E described above, but are still considered potential alternative example embodiments within the scope of this disclosure.

FIGS. 4A and 4B are diagrams of a system 400 that includes a sound processor 406, an RF transmitter 420, and a battery module 430, according to an example embodiment. The sound processor 406 may include a housing 411, an RF port 412, and an earhook 419, among various other components as described further below. As shown in FIG. 4A, the RF transmitter 420 has an RF connector 422 and an RF coil 428, and connects to the RF port 412 of the sound processor 406 via the RF connector 422.

The battery module 430 may include a housing 431, a battery 432, and an MI antenna 438. The housing 431 is an enclosure such as an aluminum casing, for example. The battery 432 may be removable, rechargeable, and replaceable. The sound processor 406 and the battery module 430 also include various circuitry (e.g., voltage, battery ID, protection components, communication components, etc.), as described above with reference to FIGS. 3B and 3C (first concept) and FIGS. 3D and 3E (second concept), and below with reference to FIG. 4C.

In some example embodiments, the housing 431 may include an extension 439 that extends below the battery 432, where the extension 439 provides extra space within the housing 431 to accommodate the MI antenna 438 below the battery 432. Preferably, the extension 439 is disposed at one side (e.g., lower left corner in FIGS. 4A and 4B) of the battery module 430 and the housing 431 that is located opposite from the RF port 412 of the sound processor 406, such that the extension 439 and hence the MI antenna 438 are located as far away from the RF coil 428 of the RF transmitter 420 as possible. It is noted that the specific size and shape of the extension 439 is not limited to the illustrative example shown in FIG. 4A, and various other configurations for the extension 439 are also possible.

Preferably, the MI antenna 438 is disposed at least 3 cm apart from the RF coil 428 that is connected to the RF port 412 of the sound processor 406. As stated above, this has been determined to be the minimum separation distance that is needed for reducing interference and ensuring good coexistence between the RF link and the MI link. In some instances, the minimum clearance required between the MI antenna 438 and the RF coil 428 may be 4 cm (e.g., for legacy implants with a combined RF link and a bilateral MI link).

FIG. 4C is a circuit diagram for the sound processor 406 and the battery module 430 of FIGS. 4A and 4B. The circuit diagram of FIG. 4C shows an MI antenna 438 connected with an interface connector 4306 of the battery module 430, and an MI transceiver 4150 connected with an interface connector 4010 of the sound processor 406. Generally, the MI transceiver 4150 on the chip should accept the parasitic components introduced by the above-described example embodiments. An “antenna trimming” process may be performed at each boot/start-up of the sound processor 406 to ensure parameter matching (i.e., between MI antenna 438 and MI transceiver 4150) every time the recipient changes the battery module 430.

FIG. 5 is a flowchart illustrating a method 500 for recalibrating MI antenna parameter(s) at each boot/start-up of a sound processor of a modular BTE device, according to an example embodiment.

At operation 510, method 500 includes booting a sound processor of a modular behind-the-ear (BTE) device. At start-up of the sound processor of the modular BTE device, method 500 further includes operations 520 and 530.

At operation 520, method 500 includes determining an identifier of a battery module connected to the sound processor, wherein the battery module includes a battery and a magnetic induction (MI) antenna.

At operation 530, method 500 includes recalibrating one or more parameters associated with the MI antenna based on the identifier of the battery module.

For example, the identifier of the battery module identifies the battery, a batch number of the battery, the MI antenna, a manufacturer of the battery or the MI antenna, or a combination thereof. Recalibrating the one or more parameters associated with the MI antenna in this manner ensures that tuning of an MI transceiver of the sound processor matches tuning of the MI antenna of the battery module.

The example embodiments described above with reference to FIGS. 2A-2D, 3A-3E, 4A-4C, and 5 provide solutions that allow a number of constraints to work together: (1) the MI antenna has to be far away from internal electrical noise (such as a DC/DC converter); (2) the MI antenna in the battery module has to be at least 3 cm/30 mm away (or 4 cm/40 mm away in some instances) from the RF coil so that the MI receiver in the sound processor is not saturated; (3) the MI receiver on the chip has to accept the parasitic components introduced by the example embodiments (e.g., refer to the circuit schematic FIG. 4C, also discussed with reference to FIGS. 3B-3E); (4) because the battery pack is modular, performing antenna trimming (parameter tuning process) at each start-up provides a way to make sure the matching is perfect every time the recipient changes the battery (e.g., refer to method 500 of FIG. 5, also discussed with reference to FIGS. 3B-3E); and (5) the battery module needs to provide enough space to place the MI antenna longitudinally within the housing (e.g., via the extension of the housing that extends below the battery, as shown in FIGS. 3A and 4A).

Advantageously, the placement of the MI antenna in the battery module reduces electromagnetic interference (EMI) originating from the RF coil in a modular BTE device. Further, the MI antenna may be placed behind the battery, in relation to the RF coil, such that the battery itself acts as an attenuator for the EMI source (the RF coil). In some example embodiments, the MI antenna may be placed on the lateral side of the battery pack, or on the downside of the battery pack, or both (i.e., at a lower corner of the battery pack). In addition to being detachable/removable, the battery pack may also be rotatable by 180 degrees (i.e., the battery module may be reversible), such that the MI antenna (e.g., a flat circular coil) is always facing tissue of the recipient. In this case, the battery pack and the MI antenna are optimized for both left side use as well as right side use by the recipient.

In some example embodiments, the MI antenna residing in the removable battery pack of the modular BTE device generates an alternating magnetic field to the implant below 30 MHz, where the alternating magnetic field provides energy to the implant. The MI antenna may also allow the battery to be charged when the battery module is removed. In addition, the alternating magnetic field may be modulated to communicate with the implant (e.g., for providing data such as sound signals, compressed audio, stimulation signals, etc.).

As mentioned above, some other modifications may include providing different locations for the MI antenna (e.g., in the ear hook, in the acoustic receiver) other than integration into the battery module, or providing additional directionality/shielding of the MI antenna. For example, ferrite materials may be included in the housing of the battery module to add further directionality to the magnetic field, and shield interfering EMI. The shape of the MI antenna could also be altered to add more directionality to the magnetic field. The solutions described above may also be used to upgrade a configurable BTE sound processor (e.g., by swapping out a conventional battery pack without an MI antenna for the battery module with the integrated MI antenna, as described above) to provide MI capability for certain markets.

### Variations and Alternatives

As previously described, the technology disclosed herein can be applied in any of a variety of circumstances and with a variety of different devices. Example devices that can benefit from technology disclosed herein are described in more detail in FIG. 6. The techniques of the present disclosure can be applied to other devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 6 illustrates an example vestibular stimulator system 602, with which embodiments presented herein can be implemented. As shown, the vestibular stimulator system 602 comprises an implantable component (vestibular stimulator) 612 and an external device/component 604 (e.g., external processing device, battery charger, remote control, etc.). The external device 604 comprises a transceiver unit 660. As such, the external device 604 is configured to transfer data (and potentially power) to the vestibular stimulator 612.

The vestibular stimulator 612 comprises an implant body (main module) 634, a lead region 636, and a stimulating assembly 616, all configured to be implanted under the skin/tissue (tissue) 615 of the recipient. The implant body 634 generally comprises a hermetically-sealed housing 638 in which RF interface circuitry, one or more rechargeable batteries, one or more processors, and a stimulator unit are disposed. The implant body 634 also includes an internal/implantable coil 614 that is generally external to the housing 638, but which is connected to the transceiver via a hermetic feedthrough (not shown).

The stimulating assembly 616 comprises a plurality of electrodes 644(1)-(3) disposed in a carrier member (e.g., a flexible silicone body). In this specific example, the stimulating assembly 616 comprises three (3) stimulation electrodes, referred to as stimulation electrodes 644(1), 644(2), and 644(3). The stimulation electrodes 644(1), 644(2), and 644(3) function as an electrical interface for delivery of electrical stimulation signals to the recipient's vestibular system.

The stimulating assembly 616 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 612, the external device 604, and/or another external device, can be configured to implement the techniques presented herein. That is, the vestibular stimulator 612, possibly in combination with the external device 604 and/or another external device, can include an evoked biological response analysis system, as described elsewhere herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A modular battery pack comprising:
a housing;
a battery disposed within the housing; and
a magnetic induction (MI) antenna integrated in the housing and disposed adjacent to the battery.

2. The modular battery pack of claim 1, wherein the MI antenna is centrally located within the housing.

3. The modular battery pack of claim 1, wherein the MI antenna is offset from a central region of the housing.

4. The modular battery pack of claim 1, wherein the housing includes an extension that extends away from the battery, wherein the MI antenna is disposed in the extension.

5. The modular battery pack of claim 1, wherein the modular battery pack is configured to connect with a behind-the-ear (BTE) sound processor via an interface connector.

6. The modular battery pack of claim 5, wherein the interface connector is configured to provide MI antenna signals to the BTE sound processor by multiplexing the MI antenna signals with other signals on one or more pins of the interface connector.

7. The modular battery pack of claim 5, wherein the interface connector is configured to provide MI antenna signals to the BTE sound processor via one or more pins dedicated for the MI antenna.

8. The modular battery pack of claim 5, wherein the BTE sound processor is configured to connect with a radio frequency (RF) coil via an RF port of the BTE sound processor.

9. The modular battery pack of claim 8, wherein the RF port is located at a first region of the BTE sound processor, and wherein when the modular battery pack is connected to the BTE sound processor, the MI antenna is located at a region of the housing that is opposite to the first region of the BTE sound processor to ensure that a threshold separation distance between the RF coil and the MI antenna is maintained.

10. The modular battery pack of claim 9, wherein the threshold separation distance provides a minimum clearance of at least 3 cm (30 mm) between the RF coil and the MI antenna.

11. The modular battery pack of claim 9, wherein the threshold separation distance provides a minimum clearance of at least 4 cm (40 mm) between the RF coil and the MI antenna.

12. The modular battery pack of claim 5, wherein the modular battery pack is detachable from the BTE sound processor, and wherein the battery is rechargeable via the MI antenna when the modular battery pack is detached from the BTE sound processor.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A device, comprising:
at least one processor;
a radio frequency (RF) coil configured to be electrically connected to the at least one processor; and
a removable battery module configured to be electrically connected to the at least one processor, wherein the removable battery module includes at least one battery and a magnetic induction (MI) antenna disposed adjacent the at least one battery.

25. The device of claim 24, wherein when the removable battery module is electrically connected to the at least one processor, the at least one battery has a first side configured to face the RF coil and a second side configured to face away from the RF coil, and wherein the MI antenna is configured to be positioned adjacent the second side of the at least one battery.

26. The device of claim 24, wherein when the removable battery module is electrically connected to the at least one processor, the MI antenna is disposed at a location within the removable battery module that ensures a threshold separation distance between the MI antenna and the RF coil.

27. The device of claim 26, wherein the threshold separation distance provides a minimum clearance of approximately 3 cm (30 mm).

28. The device of claim 26, wherein the threshold separation distance provides a minimum clearance of approximately 4 cm (40 mm).

29. A method comprising:
at start-up of a sound processor of a modular behind-the-ear (BTE) device:
determining an identifier of a battery module connected to the sound processor, wherein the battery module includes a battery and a magnetic induction (MI) antenna; and
recalibrating one or more parameters associated with the MI antenna based on the identifier of the battery module.

30. The method of claim 29, wherein the identifier of the battery module identifies the battery, a batch number of the battery, the MI antenna, a manufacturer of the battery or the MI antenna, or a combination thereof.

31. The method of claim 29, wherein recalibrating the one or more parameters associated with the MI antenna ensures that tuning of an MI transceiver of the sound processor matches tuning of the MI antenna of the battery module.
